(19) United States
(12) Patent Application Publication
(10) Pub. No.: US 2006/0020298 A1
(43) Pub. Date: Jan. 26, 2006

(54) Systems and methods for curbing appetite

(76) Inventors: Michael L. Camilleri, Rochester, MN (US); Joseph A. Murray, Rochester, MN (US); Amy E. Foxx-Orenstein, Rochester, MN (US)

Correspondence Address: MERCHANT & GOULD PC, P.O. BOX 2903, MINNEAPOLIS, MN, 55402-0903, US

(21) Appl. No.: 10/924,249

(22) Filed: Aug. 23, 2004

Related U.S. Application Data
(60) Provisional application No. 60/589,291, filed on Jul. 20, 2004.

(51) Int. Cl. A61N 1/20

(52) U.S. Cl. 607/40

(57) ABSTRACT

Methods and systems to replace and/or enhance a normal satiety signal with, e.g., an appetite control signal to curb appetite are disclosed. The appetite control signal may, e.g., enhance or accelerate the normal satiety signal and/or provide a nauseous signal in order to reduce a patient's appetite. The effector that provides the appetite control signal may function by delivering signals that are, e.g., chemical, pharmacological, electrical, biological, thermal, etc. (and combinations thereof). The system may function by sensing early physiological responses to eating using one or physiological sensors. When the physiological responses reach a predetermined level, the effector component may be activated to provide one or more selected appetite control stimuli (e.g., noxious stimuli, enhanced or accelerated satiety stimuli, etc.).

### RELATED APPLICATIONS

The entire disclosure of U.S. Provisional Patent Application 60/589,291 filed on 20 Jul. 2004, titled PHYSIOGASTRIC DEVICES AND METHODS FOR CURBING APPETITE, is incorporated herein by reference.

### BACKGROUND

There are a number of surgical methods known in the art for suppressing appetite. These include inserting objects into the upper stomach and suturing portions of the stomach to reduce its size. These methods are used to control morbid obesity because of the risks associated with such surgical procedures.

### DESCRIPTION OF THE FIGURE

FIG. 1 is a schematic diagram of one exemplary system according to the present invention depicting a physiological sensor 10 operably connected to an appetite control stimuli effector 20.

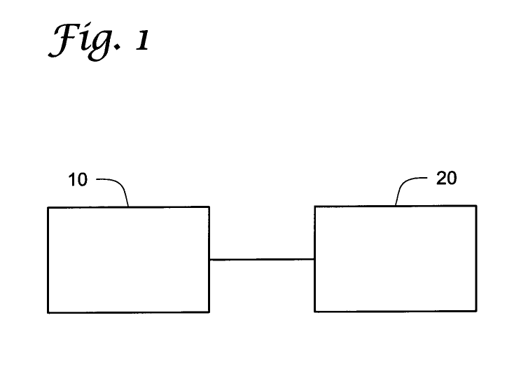

### DESCRIPTION OF ILLUSTRATIVE EMBODIMENTS OF THE INVENTION

The present invention is designed to address potential failure of the satiety response to curb appetite in patients. Results of satiety response failures may include obesity. This failure may be the result of a weak or non-existent satiety signal, the result of a failure to respond to a normal satiety signal, or the result of eating too fast for a satiety signal to work effectively.

The present invention provides systems and methods to curb appetite or otherwise provide control over weight by sensing the early onset of caloric consumption in a patient. The systems and methods can be roughly divided into two components—sensing an eating event and activating an effector to control consumption by the subject.

The present invention provides methods and systems that may preferably function to replace and/or enhance a normal satiety signal with, e.g., an appetite control signal to curb appetite. The appetite control signal may, e.g., enhance or accelerate the normal satiety signal and/or provide a nauseous signal in order to reduce a patient's appetite. The effector that provides the appetite control signal may function by delivering signals that are, e.g., chemical, pharmacological, electrical, biological, thermal, etc. (and combinations thereof).

The system would preferably function by sensing early physiological responses to eating using one or physiological sensors 10. When the physiological responses reach a predetermined level, the effector component 20 may be activated to provide one or more selected appetite control stimuli (e.g., noxious stimuli, enhanced or accelerated satiety stimuli, etc.).

It is envisioned that the appetite control signal (e.g., satiety stimuli and/or noxious stimuli) would preferably reduce a patient's desire to begin or continue eating. In some instances, it may be useful to activate the system to deliver the appetite control stimuli in the absence of physiological indications that a patient is eating. For example, the system may be activated at any time based on many different factors or at predetermined times. Activation in the absence of any physiological indications that a patient is eating may be used to, e.g., alleviate hunger.

The early physiological changes that occur in response to eating that could be measured in order to trigger the appetite control stimuli of the system may include one or more of the following: changes in pH within the stomach, changes in stomach size/tension, changes in the rate of stomach activity (e.g., contractions), changes in glucose levels of the stomach, changes in overall electrical activity of the stomach, etc. Examples of systems and methods relying on at least some of these indications may be described in, e.g., U.S. Pat. No. 6,535,764 B2 (Imran et al.).

Still other systems may operate to sense eating by sensing distension in the esophagus that may be associated with, e.g., swallowing. This detection can be accomplished using the same methodology as in stomach distension (e.g., piezoelectric elements implanted in/on esophageal wall, whose change in separation may be indicative of the passage of food). The esophagus is normally closed and, as food passes, it is distended to 0.5-2.5 centimeters (cm). Normal swallowing of saliva causes a distension roughly under 0.5 cm. Thus, the system could preferably signal an eating event if the esophagus is distended beyond approximately 0.5 cm. This signal could be combined with the number of distensions occurring over a given time (e.g., 5-6 per minute indicating an eating event versus normal swallowing of saliva of 1-2 per minute).

Sensing early eating may also be accomplished in the mouth using, e.g., a retainer-type device that fits within the mouth (along, e.g., the roof of the mouth), by sensors attached to one or more teeth, etc. A retainer device could, e.g., fit in the upper roof or lower jaw and be detachable or fixed (e.g., adhered or otherwise attached). Sensors could be attached to one or more teeth by, e.g., dental adhesives, dental restoratives, within dental implants (e.g., crowns, caps, bridges, etc.), on bands encircling one or more teeth, etc. The sensors used in a subject's mouth (on, e.g., a retainer or tooth-mounted sensor) could sense a variety of different conditions including but not limited to: temperature, pressure, pH, saliva output, presence of food or liquids (e.g., optically), etc. In some instances, it may be desirable to sense combinations of two or more different conditions, e.g., pH and pressure to reduce the likelihood of a false positive indication of eating.

If change in stomach size is used, it may be preferred to place sensors in the stomach and track changes in the distance between these sensors to measure changes in stomach size. These sensors can be placed anywhere in the stomach but are preferably placed in, near, or around the fundus. The sensors can be located inside the stomach, outside of the stomach, or both inside and outside of the stomach. The sensors may preferably be amenable to endoscopic placement and may preferably be anchored to the stomach wall. Examples of sensors that could be used are piezoelectric crystals (see the system sold by Sonometrics Corporation and U.S. Pat. No. 6,540,699 B1 (Smith)). Other physiological measuring systems and methods may be described in U.S. Pat. No. 4,154,114 (Katz et al.).

In response to the sensation of positional changes in the sensors due to the consumption of food, the system may determine that eating has begun (e.g., the separation between sensors increases, etc.). An appetite control stimulus (e.g., satiety and/or noxious stimulus) may then preferably be provided in an attempt to reduce the patient's desire to continue eating.

The noxious stimuli used in connection with the present invention may take a variety of forms. For example, the noxious stimuli may be in the form of electrical stimulation to one or more regions of the stomach (e.g., antrum, greater curve, fundus, pacer region, etc.) and/or one or more other body locations. In other instances, the noxious stimuli may be in the form of a pharmacological agent which can be dispensed into the stomach, blood stream, etc. by an implantable pump device. In some systems, different noxious stimuli may be used in combination, e.g., electrical stimulation may be used in addition to one or more pharmacological agents.

The electrical stimulation could be administered by a lead element and electrical stimulation generator (see, e.g., U.S. Pat. No. 6,327,503 B1 (Familoni) and U.S. Pat. No. 5,188,104 (Wernicke et al.). Many other implantable electrical stimulation devices and methods are known to those skilled in the art of electrical stimulation. In particular, the effector device may induce an electrical signal that modulates vagal nerve function. This modulation may include stimulation (e.g., amplification or initiation of normal vagal function) or inhibition (blocking, or preventing the vagal nerve from firing). Because the vagal nerve functions to relax the stomach to accommodate food when eating begins, it may be preferable to block vagal function to prevent stomach relaxation which may preferably limit the amount of food the stomach can hold. Examples of methods and devices that may be potentially used to modulate the vagal nerve can be found in, e.g., U.S. Pat. No. 6,611,715 ((Boveja); U.S. Pat. No. 6,609,025 (Barrett et al.); U.S. Pat. No. 6,778,854 (Puskas); U.S. Pat. No. 6,671,556 (Osario et al.); and U.S. Pat. No. 5,188,104 (Wemicke et al.).

In preparing leads to supply electrical signals to the vagal nerve, it may be preferable to separate the RF/electrical lead from the additional components necessary for function (battery/power source, microcircuitry for input-output calculations, transceivers, etc.). It may further be preferred that the RF/electrical leads be capable of endoscopic deployment. It may be preferred that the leads communicate with and receive power from the other components at a distance via a wired or wireless connection. Conventionally, RF/electrical leads used to stimulate the vagal nerve are built into a single unit with a power supply, circuitry, etc. and that requires that these units be deployed laproscopically. Although such devices may be used, it may be preferred to employ endoscopically-deployed leads.

Examples of some potentially suitable pharmacological agents may include, e.g., copper sulfate, lithium chloride, Antabuse, Antabuse plus alcohol, Apomorphine, or any other composition or combination of compositions known to result in nausea. It would be preferred that the dosage be selected in an amount effective to induce a feeling of nausea, preferably without stimulating a vomiting reflex.

It may be preferred that present invention provide a graded response to the physiological indications that a patient is eating. For example, if the indication is that eating has just begun, a measured appetite control stimulus may be delivered. As the sensors detect further eating (by, e.g., further distension of the stomach, etc.), an appetite control stimulus may be delivered that is, e.g., stronger, larger, longer, etc. may preferably be delivered. In another alternative, the graded response may involve delivery of one or more different stimuli in response to physiological indications that a patient is eating. For example, the graded response may involve an enhanced satiety stimulus at onset, followed by adding a noxious stimulus if the physiological eating indications continue.

It may further be preferred that an appetite control stimulus of the present invention be delivered relatively soon after an eating event is sensed (e.g., within 1-2 minutes after sensing the onset of an eating event). If the delay between sensing eating and delivery of an appetite control stimulus is too long, the patient may have already consumed an undesirable amount of calories.

In some embodiments, a control mechanism may be provided that would allow a patient or other individual to temporarily disable the system. The control mechanism could be accessed remotely to turn off the stimulator (see U.S. Pat. No. 6,427,088 B1 (Bowman, IV, et al.)). The control mechanism may also preferably allow a care provider to adjust the proper effective dose for a given patient if tolerated levels of, e.g., nausea, differ among the general population or change in a given patient over time.

If a pharmacologic agent (e.g., chemical, biological, etc.) located within an implanted reservoir is used to deliver the appetite control stimulus, the reservoir could be accessed endoscopically to refill the device periodically (if, e.g., the reservoir is located in the stomach). Alternatively, the reservoir could be placed subcutaneously and dispense agent to the stomach or into the blood stream. If the reservoir is placed subcutaneously, it could be refilled with a syringe transdermally. Finally, intrathecal delivery of pharmacological agents is possible in connection with the present invention. Examples of some potentially suitable implantable pharmacological agent delivery systems may be described in, e.g., U.S. Pat. No. 6,394,981 B2 (Heruth); U.S. Pat. No. 6,537,268 B1 (Gibson et al.); U.S. Pat. No. 5,976,109 (Heruth); U.S. Pat. No. 6,283,944 B1 (McMullen et al.); and U.S. Pat. No. 6,629,954 (Heruth).

It may be preferred that any implantable pharmacological delivery system may include an internal sensing mechanism to determine when agent levels are getting low, if a refill is required, or if the is any leakage of fluid occurring (see, e.g., U.S. Pat. Nos. 6,752,785 B2 and 6,461,329 B1 (both to Van Antwerp et al.)).

As used herein and in the appended claims, the singular forms “a,” “and,” and “the” include plural referents unless the context clearly dictates otherwise. Thus, for example, reference to “an anchor member” includes a plurality of anchor members and reference to “the anchor” includes reference to one or more anchors and equivalents thereof known to those skilled in the art.

All references and publications cited herein are expressly incorporated herein by reference in their entirety into this disclosure. Illustrative embodiments of this invention are discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A method for inducing satiety, the method comprising:
determining that a patient is eating; and
delivering one or more appetite control stimuli to the patient selected to curb appetite.

2. A method according to claim 1, wherein determining that a patient is eating comprises monitoring a physiological sensor attached to the patient's stomach.

3. A method according to claim 1, wherein determining that a patient is eating comprises monitoring a physiological sensor located within the patient's mouth.

4. A method according to claim 1, wherein determining that a patient is eating comprises monitoring a physiological sensor located to detect distension of the patient's esophagus.

5. A method according to claim 1, wherein determining that a patient is eating comprises monitoring two or more different physiological sensors.

6. A method according to claim 1, wherein delivering one or more appetite control stimuli comprise delivering two or more different appetite control stimuli.

7. A method according to claim 1, wherein the appetite control stimulus comprises a noxious stimulus selected to induce a feeling of nausea in the patient

8. A method according to claim 1, wherein the one or more appetite control stimuli comprise delivering one or more pharmacological agents.

9. A method according to claim 1, wherein the one or more noxious stimuli comprise delivering electrical stimulation to the stomach of the patient.

10. A system for curbing appetite in a patient, the system comprising:
one or more physiological sensors selected to indicate that a patient is eating; and
one or more implantable appetite control stimuli effectors adapted to deliver one or more appetite control stimuli to the patient;
wherein the implantable appetite control stimuli effectors are capable of being operably connected to the one or more physiological sensors to control delivery of the one or more appetite control stimuli to the patient.

11. A system according to claim 10, wherein the one or more physiological comprise a physiological sensor adapted for placement within the patient's mouth.

12. A system according to claim 11, wherein the physiological sensor adapted for placement within the patient's mouth comprises a retainer.

13. A system according to claim 11, wherein the physiological sensor adapted for placement within the patient's mouth is adapted for attachment to one or more teeth.

14. A system according to claim 10, wherein the one or more implantable appetite control stimuli effectors comprises an implantable pharmacological agent delivery system.

15. A system according to claim 14, wherein the implantable pharmacological agent delivery system comprises a pharmacological agent selected to induce a feeling of nausea in the patient.

16. A system according to claim 14, wherein the implantable pharmacological agent delivery system comprises a pharmacological agent selected to induce a feeling of satiety.

17. A system according to claim 10, wherein the one or more implantable appetite control stimuli effectors comprise at least two implantable appetite control stimuli effectors adapted to deliver different appetite control stimuli to the patient.

18. A system according to claim 10, wherein the one or more implantable appetite control stimuli effectors comprise an implantable electrical stimulus delivery system.
